(19) United States
(12) Patent Application Publication
(10) Pub. No.: US 2003/0211140 A1
(43) Pub. Date: Nov. 13, 2003

(54) Oil-core compositions for the sustained release of hydrophobic drugs

(76) Inventors: Sankaram Mantripragada, (US); Richard N. Thrift, (US)

Correspondence Address: DIANE L. GARDNER, Fish & Richardson P.C., Suite 500, 4350 La Jolla Village Drive, San Diego, CA, 92122, US

(21) Appl. No.: 10/212,030

(22) Filed: Aug. 5, 2002

Related U.S. Application Data
(63) Continuation of application No. PCT/US00/15401, filed on Jun. 2, 2000.
(60) Provisional application No. 60/137,669, filed on Jun. 4, 1999.

(51) Int. Cl. A61K 9/127; A61K 9/14; A61K 9/16; A61K 9/50

(52) U.S. Cl. 424/450; 424/490

(57) ABSTRACT

Physiologically active oil-core particles, and a method of making physiologically active oil-core particles that include a hydrophobic core material, a hydrophobic drug dissolved or suspended in the core material, and a layer of amphipathic lipids surrounding the hydrophobic core. An optional continuous phase can be an oil-immiscible solution. In one aspect, the method involves the use of a volatile solvent that is removed after the formation of the suspension. The suspension can be used substantially as created, or the particles formulated as a solid dosage form. In another aspect, the particles are formed substantially simultaneously with the volatilization of a propellant, for example, by spraying through an atomizing actuator. The resulting particles have superior particle size distribution and yield properties. The method is appropriate for use with physiologic agents that would be sensitive to heating during the encapsulating process, and also allows aseptic processing by filtration without heating the solutions used in processing.

### BACKGROUND OF THE INVENTION

The invention relates to methods of making pharmaceutical compositions that are designed to provide sustained release of drugs. These are commonly referred to as drug delivery systems.

Delivery systems for drugs offer the advantage of improved bioavailability and a higher therapeutic index over a prolonged period of time. Liposphere drug delivery vehicles have been described in U.S. Pat. Nos. 5,221,535 to Domb, 5,340,588 to Domb, 5,227,165 to Domb et al., and EP 502,119 to Domb et al. Other drug delivery vehicles referred to as emulsomes have been described in U.S. Pat. No. 5,576,016 to Anselem et al. U.S. Pat. No. 5,672,358 to Tabibi et al. provides another example of a drug delivery vehicle. Other drug delivery vehicles are described in EP 605,497 B1 to Medac Gesellschaft fir Klinische Speczialpräparate GmbH, and in Müller et al., Eur. J. Pharm. Biopharm., 41, 62-69 (1995). The compositions disclosed in these references have solid lipid cores. These compositions have been prepared by a number of different methods. For example, the solid core material has been melted along with the drug to be delivered. Volatile solvent has not been used in such processes. In another example of solid core particle preparation, a volatile solvent is used in early stages of production, but removed before the addition of an aqueous phase, so that the drug delivery vehicles are harvested and dried before the addition of an aqueous continuous phase.

Liquid core particles have also been prepared for use in drug delivery applications. These preparations have either involved processes in which volatile solvent is not used (for example, U.S. Pat. No. 5,514,673 to Heckenmüller et al., U.S. Pat. No. 5,637,317 to Dietl, or U.S. Pat. No. 5,877,205 to Andersson), or processes in which volatile solvent is removed before the addition of an aqueous phase (including U.S. Pat. No. 4,298,594 to Sears et al. and U.S. Pat. No. 5,616,330 to Kaufman et al.). In another variation of liquid core particle production, the liquid core material is extruded into an aqueous phase to produce a drug delivery system, as described by U.S. Pat. No. 4,610,868 to Fountain et al. Reverse osmosis has also been employed to remove water-miscible solvent in preparing drug delivery systems, as disclosed in U.S. Pat. No. 4,994,213 to Aitcheson et al. Aerosolized formulations using glycerol phosphatides without oil are described in U.S. Pat. No. 4,814,161 to Jinks et al., and aerosolized formulations using C₁₆₊ unsaturated vegetable oil to prevent aggregation of the medicament without surfactant are described in U.S. Pat. No. 5,635,161 to Adjei et al.

### SUMMARY OF THE INVENTION

The invention provides methods for making physiologically active oil-core particles for sustained release. The particles include an oil core into which a drug is dissolved or suspended, and at least one type of amphipathic surfactant coating the core. The particles can be formulated as a suspension in an oil-immiscible liquid, can be made in a dried form, or can be prepared in situ by means of a volatile propellant. The latter method can form the basis of an aerosol delivery method for the sustained release particles disclosed herein. In the inventive methods, the oil-core phase initially includes a volatile solvent, which can be removed after a suspension is produced. The solvent removal can optionally involve the use of a propellant that volatilizes upon spraying. Any of these methods can be used to produce a high process yield, and a high loading of drug in the particles. A very homogeneously dispersed suspension of such particles can be produced by the inventive methods, or, when a propellant is used, particles can be sprayed into or onto an aqueous phase, or on a solid surface.

The invention allows the preparation of oil-core particles having a superior process yield and loading of drug within them. The particles have relevant properties that are superior to particles prepared without the use of a volatile solvent, as well as to particles prepared by a method in which a volatile solvent is removed before the addition of an oil-immiscible phase.

The pharmaceutical compositions of the present invention also afford release of drug in vivo over a sustained period, to provide beneficial effects in the treatment of, diagnosis of, or prophylaxis against, an undesired condition in an individual. Sensory and motor block effects produced in test subjects by drugs determined at various times show that the inhibition of such responses peaked at a later time, and persisted longer for the inventive pharmaceutical compositions than was the case for the same drugs not present in particles. In vivo pharmacokinetic analysis demonstrates increased exposure to drugs administered via the pharmaceutical compositions of the invention. Alternately, those oil-core particles containing drugs which inhibit the release of endogenous serum components (for example, hormones, enzymes, proteins, carbohydrates and the like) can show a decrease in the serum concentration of the inhibited component which is longer lasting than that observed for drugs not contained within particles. The sustained release allows a convenient means of administration, and can be far less invasive than a more continuous route of administration for many drugs.

One objective of the present invention is to provide a novel pharmaceutical composition as a suspended oil-core particle drug-delivery system with a drug encapsulated within the particle core, the composition enabling release of the agent over a prolonged period of time. Another objective is to provide a means of modulating the rate of release of the agent from the particles. Another objective is to provide a means of formulating and storing the composition either as a solid dosage form or a semi-solid dosage form.

Preparation of drug delivery systems according to the prior art typically requires that a volatile solvent, when used at all, be removed prior to formation of particles that occurs upon suspension of the hydrophobic phase in an aqueous solution. The oil-core particles of the present invention are made with a volatile solvent and/or propellant included in their hydrophobic phase. The volatile solvent used in the inventive method can be removed from the suspension after the introduction of an oil-immiscible phase and concomitant particle formation, providing a superior product, as disclosed herein. Removal of solvent from this suspension can be by sparging, or by pressure reduction over the suspension. Alternatively, volatile solvent (propellant) can be removed upon forming particles by spraying the hydrophobic phase containing a volatile gaseous or liquid propellant without the introduction of any oil-immiscible phase. Higher yield and a greater loading of the drug are obtained for the pharmaceutical oil-core particles of the present invention than for drug-delivery systems of the prior art.

In one aspect, the invention provides physiologically active oil-core particles, where each particle includes a hydrophobic oil core including at least one triglyceride, and a hydrophobic drug; and at least one amphipathic surfactant. The particles can have a median diameter of from about 0.5 to about 30 microns, with a standard deviation of the particle diameters of from about 0.1 to about 15 micronsor from about 0.1 to about 10 microns. The oil core can be liquid or solid at ambient temperature.

In another aspect, the invention provides a method of making physiologically active oil-core particles. The method includes mixing 1) a hydrophobic solution including at least one hydrophobic oil material; a drug, wherein the drug is soluble in the oil material; at least one amphipathic phospholipid; a volatile organic solvent; and optional constituents, with 2) an aqueous solution, to form a suspension of physiologically active oil-core particles. In another step, the method includes removing the volatile organic solvent from the suspension to form a substantially solvent-free suspension of physiologically active oil-core particles. The particles can have a liquid or solid oil core at ambient temperature. The oil material can include at least one triglyceride having fatty acid chains selected from butyric acid, caproic acid, caprylic acid, capric acid, lauric acid, lauroleic acid, myristic acid, myristoleic acid, pentadecanoic acid, palmitic acid, palnitoleic acid, margaric acid, stearic acid, oleic acid, linoleic acid, linolenic acid, ricinoleic acid, dihydroxystearic acid, licanic acid, eleostearic acid, arachidic acid, eicosenoic acid, eicosapolyenoic acid, behenic acid and erucic acid. The amphipathic phospholipid can be a phosphatidic acid, phosphatidylserine, phosphatidylglycerol, phosphatidylinositol, cardiolipin, phosphatidylcholine, phosphatidylethanolamine, or sphingomyelin. The optional constituents can be diacyl dimethylammonium propanes, acyl trimethylammonium propanes, stearylamine, cholesterol, ergosterol, nanosterol, and their esters. The aqueous solution can include water and at least one pharmaceutical excipient, which can be amino acids, sorbitol, mannitol or sugars. The drug can be oil-phase soluble derivatives of semisynthetic amino glycoside antibiotics, antidiabetics, peptides, antitumor drugs, antineoplastics, alkaloid opiate analgesics, local anesthetics, synthetic anti-inflammatory adrenocortical steroid, antimetabolites, glycopeptide antibiotics, vincaleukoblastines, stathmokinetic oncolytic agents, hormones, cytokines, or growth factors. For example, the oil-phase soluble derivatives of paclitaxel, morphine, hydromorphone, bupivacaine, dexamethasone, vincristine and vinblastine, such as bupivacaine free base, or paclitaxel. The particles can be made to release the drug with a half time of at least 10 hours, 20 hours, or 40 hours. The particles can have a median diameter of from about 0.5 to about 30 microns, with a standard deviation of the particle diameter of from about 0.1 to about 15 microns, or from about 0.1 to about 10 microns. The mixing can be carried out with a high-speed shear mixer.

In a particular embodiment, the hydrophobic drug can be paclitaxel, the hydrophobic oil core can be tributyrin, and the amphipathic surfactants can be dipalmitoyl phosphatidylglycerol, dioleoyl phosphatidylcholine and cholesterol. In another particular embodiment, the hydrophobic drug can be bupivacaine, the hydrophobic oil core can be tricaprylin, and the amphipathic surfactants can be dipalmitoyl phosphatidylglycerol, dioleoyl phosphatidylcholine and cholesterol.

In another aspect, the invention provides a method of making bupivacaine-containing oil-core particles. The method includes mixing 1) a hydrophobic solution including bupivacaine free base; tricaprylin; dioleoylphosphatidylcholine, and dipalmitoylphosphatidylglycerol; chloroform; and cholesterol, with 2) an aqueous solution including 5 mM lysine, to form a suspension of bupivacaine-containing oil-core particles. In another step, the method includes removing the chloroform from the suspension to form a substantially chloroform-free suspension of physiologically active oil-core particles. The method can carried out as an aseptic process.

In another aspect, the invention provides a substantially solvent-free physiologically active suspension made by the methods disclosed herein.

In yet another aspect, the invention provides a pharmaceutical composition including such substantially solvent-free physiologically active suspensions.

In yet another aspect, the invention provides a method of treating, diagnosing, or providing prophylaxis against an undesired condition in an individual, the method including administering a pharmaceutical composition described herein.

In yet another aspect, the invention provides a method of providing anesthesia to an individual in need of anesthesia, by administering a pharmaceutical composition including bupivacaine-containing particles made according to the methods described herein.

In yet another aspect, the invention includes a method of making physiologically active oil-core particles. The method includes mixing 1) a hydrophobic solution including at least one hydrophobic oil material; a drug that is soluble in the oil material; at least one amphipathic phospholipid; and optional constituents, with 2) a volatile propellant. In another step, the method includes allowing volatilization of the propellant to form a substantially solvent-free preparation of physiologically active oil-core particles. The volatilization can takes place through an orifice of size appropriate to form physiologically active oil-core particles having a median diameter of from about 0.5 to about 30 microns. The physiologically active oil-core particles can be deposited to contact an oil-immiscible phase, such as an aqueous phase. The aqueous phase can include pharmaceutically acceptable adjuvants. The propellant can be a fluorinated hydrocarbon, or chlorofluorohydrocarbon, or mixtures thereof. The volatilization can produce an aerosol containing physiologically active oil-core particles in a quantity sufficient to produce a physiological effect. The drug can be, for example, paclitaxel or bupivacaine.

In yet another aspect, the invention provides a method of administering physiologically active oil-core particles to a subject. The method includes a) formation of an aerosol of physiologically active oil-core particles, b) volatilization of a volatile propellant, and c) allowing contact of the aerosol with the subject. In particular embodiments, the hydrophobic drug can be paclitaxel, the hydrophobic oil core can include tributyrin, and the amphipathic surfactants can be dipalmitoyl phosphatidylglycerol, dioleoyl phosphatidylcholine and cholesterol. In another particular embodiment hydrophobic drug can be bupivacaine, the hydrophobic oil core can include tricaprylin, and the amphipathic surfactants can be dipalmitoyl phosphatidylglycerol, dioleoyl phosphatidylcholine and cholesterol.

The term “oil” as used throughout the specification and claims refers to oils, fats, waxes and other hydrocarbon materials, all being essentially hydrophobic in nature. The “particles” of the present invention can be spherical or approximately spherical, but need not be of any particular shape to be effective in their function. The term “suspension” as used throughout the specification and claims includes a mixture of two or more immiscible liquids, one being present in the other in the form of droplets. In the present invention, the suspension comprises hydrophobic droplets (a dispersed phase) dispersed throughout an aqueous phase (a continuous phase). A variation in which molten waxes or fats are dispersed in an oil-immiscible phase is also included in the definition of “suspension”. The term “oil-core particles” as used throughout the specification and claims refers to the hydrophobic droplets, which are coated with at least one surfactant layer. These particles can be used in pharmaceutical compositions of the invention.

The term “drug” as used throughout the specification and the claims, refers to physiologically active agents of all kinds, including those specifically noted herein. The term “releasable from the particles” refers to the condition that upon sufficient partitioning of the drug from the particles, or upon sufficient biodegradation of the particles, the drug (encapsulated within, or on the surface of, the particles) is able to exert its physiological effect. Implicit in the definition is the idea that when the agent is not released, its effect is diminished to the extent that a physiological effect is not observable. The drug can be released from not only the interior of a particle, but also from the particle wall. It is further to be understood that a drug, to be useful in the present invention, exists in a form which allows solubilization in the oil cores of the particles of the invention. Thus, if a drug has a protonatable group, such as is the case for an amine-containing drug, for example, this group will not be protonated to give the group a positive charge. Thus, such a drug should be present in its free base form. Similarly, groups which are deprotonatable to give the group a negative charge, such as carboxylic acid groups, for example, will not be deprotonated, but should exist in their free acid form. For zwitterionic drugs, the protonatable/deprotonatable groups shall be present in their net uncharged forms. The term “therapeutically effective” as it pertains to the compositions of this invention, means that a drug present in the particles is released in a manner sufficient to achieve a particular level of treatment of a disorder.

The term “ambient temperature” includes temperatures generally found in reasonably controlled environments of interior spaces in laboratories, work spaces, and commercial establishments, which typically ranges from about 18 to about 25□C. The terms “an oil core which is liquid at ambient temperature” and “an oil core that is solid at ambient temperature” refer to the core of the particles as loaded with drug. The term “propellant” as used herein, refers to pharmacologically inert liquids with boiling points from about −30° C. to about 25° C., which singly or in combination exhibit a high vapor pressure at about 25° C. The term “sparging,” as used herein, refers to the passage of a non-reactive gas, such as nitrogen, through a solution or suspension in order to remove a volatile component of the solution or suspension by partitioning the volatile component into the gas phase.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only, and not intended to be limiting. Other features and advantages of the invention will be apparent from the following detailed description, and from the claims.

### BRIEF DESCRIPTION OF THE DRAWINGS

FIG. 1 is a graph showing the duration of sensory block versus time after administration of pharmaceutical compositions including physiologically active oil-core particles, drug not present in particles, and control measurements.

FIG. 2 is a graph showing the duration of motor block versus time after administration of pharmaceutical compositions including physiologically active oil-core particles and drug not present in particles.

FIG. 3 is a graph showing the negative response to stimuli versus time after administration of pharmaceutical compositions including physiologically active oil-core particles and drug not encapsulated in particles.

FIG. 4 is a graph showing in vivo drug concentrations versus time after administration of pharmaceutical compositions including drug formulated in oil-core particles and the conventional formulation of the drug.

### DETAILED DESCRIPTION

The invention provides physiologically active oil-core particles, and methods for making physiologically active oil-core particles. The methods involve making particles which contain an oil core into which a hydrophobic drug or drug modified to be hydrophobic is dissolved or suspended. The hydrophobic core is surrounded by at least one type of amphipathic surfactant. The invention is based on the finding that methods of making such particles can include the use of a volatile organic solvent, and that the removal of that solvent subsequent to the formation of particles, that is, subsequent to the dispersion of the hydrophobic core into a continuous oil-immiscible phase can produce particles of superior physical and functional properties. In another aspect, the invention involves the in situ formation of physiologically active oil-core particles using a propellant. As used herein, the term “in situ” refers to the formation of physiologically active oil-core particles substantially simultaneously with the volatilization of propellant, for example, through an actuator. The particles thus formed can be subsequently deposited in an oil-immiscible phase such as an aqueous phase, or on a surface, such as a mouth, tonsil or lung surface. These methods of preparation result in physiologically active oil-core particles that have superior performance characteristics with respect to particle size, size distribution, and product yield when compared with those of the prior art.

The particles can be suspended in an oil-immiscible phase. In one aspect, the method generally involves the mixing of a hydrophobic phase with an oil-immiscible phase, for example, an aqueous phase, to produce droplets of the hydrophobic phase. The hydrophobic phase includes a volatile solvent that is removed from the mixture after the droplets are formed. The particles formed this way comprise oil-core particles suspended in an oil-immiscible phase. The pharmaceutical preparation that results from this method does not need to be reconstituted from a dry product. An associated method produces particles that are not suspended in a continuous phase, for example, a lyophilized formulation of such particles.

In another aspect, the method generally involves evaporation of a propellant rapidly upon formation of droplets, such as, for example, by forcing the hydrophobic phase through an orifice. The particles formed this way can be deposited into, or onto, an oil-immiscible phase, or alternatively, onto a solid surface. Thus, the method can be used for aerosol delivery of sustained-release particles.

The hydrophobic phase comprises at least one hydrophobic drug, a hydrophobic core constituent, and a volatile solvent. Optionally, the phase includes a propellant that is able to readily volatilize at atmospheric pressure, but is a liquid at pressures that are easily attained in industrial scale pharmaceutical production. The propellant can serve as volatile solvent, or can be a co-solvent. The volatile solvent or propellant is substantially removed from the composition at a point after formation of droplets or particles of hydrophobic phase. Alternatively, the volatile solvent is substantially removed from the composition essentially simultaneously with formation of the oil-core particles, an aqueous phase not being necessary to the formation or stability of the particles. The hydrophobic phase can also contain at least one amphipathic surfactant in an amount sufficient to provide a substantially complete coating on the surface of the core. Alternatively, the amphipathic surfactant can be used in the oil-immiscible phase.

The use of a volatile solvent and/or propellant is desirable, since in the absence of solvent the surfactants and other components of the core coating are more difficult to solubilize, and the solutions tend to become turbid. Without a solvent and/or propellant, it may be necessary to maintain the preparation at an elevated temperature in order to keep the components in solution, or heat may be required to speed up dissolution of the components. If the components are not kept solubilized, it is more difficult to ensure that all components eventually become distributed uniformly throughout the product batch, to ensure complete transfer between vessels, or to sterilize by filtration. The presence of solvent and/or propellant lowers viscosity and makes the fluid easier to handle, with less residue remaining on surfaces. The presence of solvent allows production of particles with a narrower size distribution, and a better control of particle size. The individual components of the hydrophobic phase are discussed in more detail in the following sections.

### Hydrophobic Core Material

The oil-core particles of the invention have as their centers a core that includes a hydrophobic material. Thus, an essential constituent of the material making up the hydrophobic phase is such a hydrophobic core material. This hydrophobic material acts as a carrier vehicle for hydrophobic drugs. The hydrophobic core materials of the inventive particles can be oils, fats, waxes or other materials to be described in this section. The essential requirement for the hydrophobic core material is that any drug that is to be utilized in the particles of the present invention must be able to be suspended or dissolved in the core material. In some embodiments of the invention, the hydrophobic core material includes solid or liquid oils. Some core materials that are useful in the practice of the invention are liquid at ambient temperature only upon mixture with a drug.

In some of the preferred embodiments of the invention, the oils are liquid at ambient temperature and pressure. The use of a liquid hydrophobic core material can offer the advantage that there is less concern for nonuniform size dispersion in the inventive particles than there would be with a solid hydrophobic core material. The use of solid hydrophobic core particles may require the solid material to be melted in order that drug be homogeneously dispersed throughout the core, then the core material is cooled to regain its solid state. Some physiologically active agents may not be stable and may become permanently inactivated at the elevated temperatures needed to melt solid core materials. Equipment may be easier and cheaper to design and build if provision for heating is not required. Attempting to trap a drug in a solid matrix can lead to inadequate sequestration of the drug, manifested in low yields or in an undesirably rapid release (burst effect).

Currently, solubility of drug in the liquid core is a limiting factor for liquid-core particles, although it is likely that in the future emulsifiers can be found which will enable sols or aqueous solutions to be adequately suspended within the liquid core. Thus, solid-core particles may in some cases achieve higher drug loading than liquid-core particles, and are not necessarily limited to encapsulating those drugs that are soluble in lipids at room temperature.

Mixtures of oils can be used in the cores of the inventive particles. This includes mixtures of oils wherein the individual oils are not either liquid or, in alternative embodiments, solid at the desired temperature, but the resulting mixture is liquid or, in alternative embodiments, solid at the desired temperature. Liquid core materials require that a drug be used at a concentration below the solubility limit. The liquid oil core could be heated to allow the concentration of drug to be increased, but in some cases, the drug can be heat sensitive. It is considered desirable to avoid the formation of crystals of drug in the core material of the present particles. Such phenomena can result in a product that has a greater or lesser amount of crystallized drug, depending on the amount of time the product has been stored. This is undesirable from the point of view of uniform administration of the inventive compositions, since product with a variable amount of crystalline drug can have a variable physiological response.

The hydrophobic core materials that can be included in the particles of the invention include triglycerides (triacylglycerols). Such triglycerides can be those with saturated, unsaturated, and multiply unsaturated acyl chains. The acyl chains are fatty acid chains that can be esterified onto glycerol, or those that are naturally occurring. The chain lengths of the acyl chains can range from about 4 carbons to about 22 carbons. Exemplary fatty acids which can be incorporated into the triglycerides of the liquid oil cores include butyric acid, caproic acid, caprylic acid, capric acid, lauric acid, lauroleic acid, myristic acid, myristoleic acid, pentadecanoic acid, palmitic acid, palmitoleic acid, margaric acid, stearic acid, oleic acid, linoleic acid, linolenic acid, ricinoleic acid, dihydroxystearic acid, licanic acid, eleostearic acid, arachidic acid, eicosenoic acid, eicosapolyenoic acid, behenic acid and erucic acid. The three fatty acid chains can be all the same or not all the same. For example, triolein, tricaprylin, tributyrin, tricaprin, tricaprylolein, tripalmitolein, trilinolein, trilinolenin, fractionated vegetable oils (sesame, soy, coconut), and structured lipids (including mixed-chain triglycerides such as CAPTEX®) are useful as oil cores for the particles of the invention.

The oils can be synthetic or naturally occurring. Some naturally occurring oils that are useful in the practice of the invention include babassu, butterfat, castor, cocoa butter, coconut, corn, cottonseed, herring, lard, linseed, menhaden, mustard seed, neatsfoot, oiticica, olive, palm, palm kernel, peanut, perila, rapeseed, rice bran, safflower, sardine, sesame, soybean, sunflower, tallow, and tung oil. Other materials such as mineral oil, fluorinated hydrocarbons, vitamin E acetate, diglycerides, and squalene can also be used as the liquid oil core.

Solid hydrophobic core materials that can be used in the particles of the invention include natural, regenerated or synthetic waxes including carnuba wax, cetyl palmitate, cera alba and beeswax; steroidal materials such as cholesterol and cholesteryl palmitate; fatty acid esters such as ethyl stearate, isopropyl palmitate, and isopropyl myristate; fatty alcohols such as oleyl alcohol, cetyl alcohol, stearyl alcohol, and cetostearyl alcohol; solid oils; paraffinic materials; and hard fat such as tristearin.

These materials can be present in amounts of from about 0.1 mg/mL to about 900 mg/mL. Alternatively, these materials are present in amounts of from about 75-750 mg/mL.

### Hydrophobic Drug

The oil-core particles of the invention include a hydrophobic drug dissolved in, or suspended in, the oil core. In some embodiments of the invention, the drug can be incorporated into the surfactant coating, as well as, or instead of, the core, or subsequently adhered to the surfactant coating, by selection of surfactant and drug having appropriate chemical properties.

Hydrophobic drugs can be difficult to deliver to their sites of action unless they are carried in a hydrophobic medium, such as that used in the present invention. Further, even for hydrophobic drugs that have significant (though limited) solubility in water (such as bupivacaine), administering the drug in colloidal form presents the opportunity for controlled or sustained release.

At physiological pH values, some drugs exist in a salt form. Such ionized forms are not particularly stable in a hydrophobic environment, and will partition out of the core regions of an oil-core particle, and into the surrounding oil-immiscible phase. This results in a net loss of encapsulated drug, to the detriment of the efficacy of the invention. For those cationic drugs which have a pKₐ which is higher than physiological pH, it can be advantageous to produce the suspended particles in an aqueous phase having a pH which is above the pKₐ of a drug, but not so high so as to cause irritation upon parenteral administration. For those acidic drugs that have a pKₐ which is lower than physiological pH, it can be advantageous to prepare the suspended particles in an aqueous phase having a pH which is below the pKₐ of a drug, but not so low so as to cause irritation upon parenteral administration. A preferable pH range for suspensions including the particles of the invention is from about 2.0 to about 10. Such preparations have the advantage that the drug does not tend to become converted to its salt form, and does not thereby tend to become more soluble in the continuous aqueous phase than in the core of the particles. It is therefore considered desirable that the drugs to be used in the hydrophobic core of the inventive particles be present in their free acid or free base forms. If non-aqueous oil-immiscible phases are utilized, it is equally important that the drug not readily partition out of the particle core due to unstable interactions between it and the core material. The drug should desirably undergo sustained release primarily according to a partition of the drug out of the particles effectuated by exposure of the particles to physiological pH. The drug can also be released by physiological breakdown of the particles themselves, for example, through the action of enzymes, although this is not believed to be the primary mode of release.

A wide variety of drugs can be employed in the inventive pharmaceutical preparations, including antianginas, antiarrhythmics, antiasthmatic agents, antibiotics, antimicrobials, antidiabetics, antifungals, antihistamines, antihypertensives, antiparasitics, antineoplastics, antivirals, cardiac glycosides, herbicides, hormones, immunomodulators, neurotransmitters, proteins, radio contrast agents, radio nuclides, sedatives, anxiolytics, antidepressants, anticonvulsants, analgesics, nonsteroidal anti-inflammatory drugs, steroids, anticholinersterases, tranquilizers, vaccines, vasopressors, general and local anesthetics, hypnotics, peptides, and combinations thereof.

Of particular interest are semisynthetic amino glycoside antibiotics; antidiabetics; peptides; antitumor drugs such as paclitaxel and camptothecin; antineoplastics such as doxorubicin, ; alkaloid opiate analgesics including morphine and hydromorphone; antifungals such as griseofulvin; antifungal triazoles such as filuconazole; polyene antibiotics such as amphotericin B; anesthetics such as propofol and etomidate; local anesthetics such as bupivacaine; cephalosporins; prostaglandins; leukotrienes; retinoids such as all-trans retinoic acid, 9-cis-retinoic acid, retinoic acid alpha-tocopheryl ester; cholinergics such as pilocarpine; anticholinergics such as scopolamine; synthetic anti-inflammatory adrenocortical steroids including dexamethasone; nonsteroidal anti-inflammatories such as indomethacin; antipsychotics such as haloperidol decanoate; antihypertensives such as levobunolol, timolol; and anticonvulsants such as phenytoin; antimetabolites; glycopeptide antibiotics; vincaleukoblastines and stathmokinetic oncolytic agents including vincristine and vinblastine; hormones; cytokines; growth factors. Prodrugs that undergo conversion to the indicated physiologically active substances upon local interactions with the intracellular medium, cells or tissues can also be employed in the invention.

The drugs can be used alone, or in combination with the limitation that the amount of the substance in the resulting pharmaceutical composition be sufficient to enable the diagnosis of, prophylaxis against, or the treatment of, an undesired condition in a living being.

The drugs are present in amounts of from about 1 fg/mL to about 750 mg/mL. Preferably, the drugs are present in amounts of from about 0.1 mg/mL to about 750 mg/mL.

### Amphipathic Surfactant

The oil-core particles of the invention include a coating of amphipathic surfactant forming a layer around the hydrophobic core and drug. This coating can be a monolayer, or more than a monolayer. The particles will generally be structured according to a configuration that produces a monolayer of amphipathic surfactant on the surface of the core, as this is typically the lowest energy configuration. In some preferred embodiments, the core will be at least substantially, if not completely, coated with amphipathic surfactant. The surfactants can be natural or synthetic in origin and can include lipids such as phospholipids, sphingolipids, sphingophospholipids, sterols and glycerides. These amphipathic materials generally have a polar “head” group and a hydrophobic “tail” group, or as in the case of block copolymers can have alternating hydrophilic and hydrophobic regions, and can have membrane-forming capabilities. The phospholipids and sphingolipids can be anionic, cationic, nonionic or zwitterionic (having no net charge at their isoelectric point), wherein the hydrocarbon chains of the lipids are typically between 12 and 22 carbon atoms in length, and have varying degrees of unsaturation. In the inventive particles, the polar head groups of a phospholipid-type surfactant will be at the interface between the microsphere interior and the oil-immiscible phase, and the hydrophilic tail will extend into the hydrophobic core of the particles.

Amphipathic phospholipids are based on a parent structure of diacylglycerolphosphate having an organic moiety attached to the phosphate. The acyl groups are based on fatty acids including those having chain lengths from about 4 carbons to about 22 carbons, and which can further be saturated, unsaturated or multiply unsaturated chains. The fatty acids of the diacylglycerolphosphate can be the same or different. They may also be joined to each other covalently or ionically, to effectively form a single difunctional group bridging the glycerol.

The organic moieties that are attached to the phosphate groups of the amphipathic phospholipids include choline, ethanolamine, inositol, serine, glycerol, and sphingosine. Preferred anionic phospholipids include phosphatidic acids, phosphatidylserines, phosphatidylglycerols, phosphatidylinositols and cardiolipins. Preferred zwitterionic phospholipids include phosphatidylcholines, phosphatidylethanolamines, and sphingomyelins. Preferred cationic lipids include diacyl dimethylammonium propanes, acyl trimethylammonium propanes, and stearylamine. Preferred sterols include cholesterol, ergosterol, lanosterol, and esters thereof. The glycerides can be monoglycerides or diglycerides.

Suitable amphipathic phospholipids for use in the particles of the invention include phosphatidylcholines, such as dioleoylphosphatidylcholine (DOPC); phosphatidylethanolamines; phosphatidylinositols; phosphatidylserines; phosphatidylglycerols, such as dipalmitoylphosphatidylglycerol (DPPG); and phosphatidylsphingosines. Naturally occurring phospholipid-containing materials such as lecithin can also be successfully used in the particles of the present invention.

Other useful surfactants include nonionic surfactants such as block copolymers of alkylene oxides, including block copolymers of propylene oxides and ethylene oxides, commercially available as PLURONIC® surfactants (BASF Corp.); sorbitan-derived lipids, including sorbitan mono-, di- and tri-fatty acid esters, where the fatty acids are selected from C₁₀-C₂₀ saturated and unsaturated acids, commrcially available as SPAN® surfactants (ICI Americas, Inc.); and polyoxyethylene sorbitan-derived mono-, di- and tri-fatty acids esters, commercially available as TWEEN® surfactants (ICI Americas, Inc.). The surfactants can be present in an amount of from about 100 ng/mL to about 100 mg/mL by weight, based on the total.

The components of the hydrophobic phase are mixed until they are homogeneously distributed. This mixing can be carried out by any of a number of known mixing methods, including the use of a high-speed homogenizer, static mixer, or other means of mixing.

### Oil-Immiscible Phase

The oil-core particles of the invention can be produced in an oil-immiscible continuous phase if desirable. This phase is typically an aqueous phase, and further, is generally mostly water, preferably deionized water. Other ingredients which can be found in the aqueous phase are those such as pharmaceutical excipients such as ionic species, thickening agents, buffering agents, acids or bases for pH adjustment, antifoam agents, antioxidants, chelators, emulsifiers, preservatives, suspending agents, stabilizing agents, tonicity agents, and viscosity-adjusting agents. These excipients include sugars, sugar alcohols, especially glucose, mannose, trehalose, mannitol, sorbitol, as well as amino acids, or salts (for example, sodium chloride), including alkali or alkali metal salts of citrate, pyrophosphate, or sorbate. Other excipicnts that are not necessarily in the aqueous phase include surfactants, emulsifiers, and antioxidants. Such optional components can be present in an amount of from about 0.01 mM to about 500 mM, preferably from about 0.1 mM to about 320 mM.

### Producing a Suspension

In one aspect, the hydrophobic and oil-immiscible phases are mixed together to form a suspension of oil-core particles. The means for creating this suspension can be a high speed mixer, a homogenizer, a static mixer, a sonicator, or by passing the hydrophobic phase through a syringe needle, porous pipe, or other means for producing substantially uniform particles into the aqueous phase. The mixing is carried out until the particles have been reduced to the appropriate size.

Particle size can be generally controlled by the energy input into emulsification, the components used, the volume fraction of hydrophobic and oil-immiscible phases, but in general will be from about 20 run to about 200 microns. The viscosity of the emulsion can be used as a process parameter to indicate particle size, as described in commonly owned U.S. patent application Ser. No. 09/192,064, hereby incorporated by reference in its entirety. During mixing, the droplets of the discontinuous phase are deformed due to the shear exerted until the shear forces exceed the surface tension forces. At this point, the droplets are broken into smaller droplets. For a given oil and oil-immiscible phase system, the quality of the emulsion is controlled by the volume fraction of each phase, temperature and mixing speed and time. In addition, the ratio of amphipathic liquid to hydrophobic phase, and the choice of the vessel and shear device will affect the emulsion as well.

The characteristics of the emulsion step can be determined by phase separation in a gravimetric field, droplet size distribution, emulsion viscosity, and conductivity of the continuous phase. Different droplet sizes are obtained by varying the emulsification method (for example, by adjusting the impeller speed in a shear mixer) and temperature.

The volatile solvent is removed after the addition of, and emulsification with, the oil-immiscible phase. This can result in a superior suspension of particles, since in prior art processes which include removal of a solvent before addition of an aqueous phase, the ability to homogeneously disperse the solid mass of particles obtained after solvent evaporation can be impaired by the stickiness of the particles. The present process, in which a volatile solvent is employed, and removed after the addition of an oil-immiscible phase, produces a superior dispersion of particles, particularly with regard to homogeneity.

Volatile solvents that can be used in the production of oil-core particles include any which are immiscible with water and can be readily removed by sparging, or by reduction in pressure over the suspension. Mild heating can be employed in particular circumstances that do not result in undue damage to the particles or their contents. Preferred are those solvents that are not hazardous by reason of flammability or environmental damage. For example, chloroform, methylene chloride, propyl propionate, or isopropyl ether can be used.

Solvent removal can be accomplished by sparging with a gas, such as air, nitrogen, argon, or another gas that does not significantly react with the particles or otherwise disrupt their structures. The rate of gas sparging can be important. Solvent removal should be done in a manner that does not remove too much water from the suspension. The suspension may become too concentrated, resulting in coalescence, aggregation, or difficult handling. In some cases, maintaining the right osmolarity may be important, in others the pH or other parameter may drift out the desired range if too much water is removed. Conceivably, particles may be more likely to coalesce before solvent removal, so that the solvent should be removed fast enough to minimize such rearrangement. The rate of solvent removal may depend among other things on the vapor pressure of the solvent, the solubility of the solvent in the aqueous phase, and the partitioning of solvent between particles and the aqueous phase. Solvent removal can also be accomplished by reducing the pressure over the suspension. If pressure is reduced to the point where boiling or cavitation of either the solvent or the aqueous phase occurs, the particles may be disrupted. Solvent removal is continued until the solvent is brought to levels that are acceptable in terms of toxicity limits and that do not lead to significant coalescence of particles. The limit for chloroform currently is preferably below 50 ppm. The inventive methods of preparation do not require heating the liquid phases to temperatures higher that those generally useful for the removal of volatile solvents, that is, 37-45° C. Thus, the inventive methods can be used to prepare drug-containing particles wherein the drugs are sensitive to temperatures higher than about 37-45° C.

The resulting product is an aqueous suspension of physiologically active particles having an oil core with amphipathic surfactants and optionally, other constituents. The size of the oil-core particles can range from about 20 nm to about 200 microns. Preferably, the particles range in size from about 0.5 to about 50 microns for non-intravenous administration, for example, between about 0.5 and 20 microns for endopulmonary or nasal administration. For intravenous administration, preferably the particles range from about 20 to 1000 nanometers. The density of particles in the aqueous phase can range from about 0.5 to about 2.2 g/mL.

### Propellant-Based Method

As an alternative to forming a suspension of oil-core particles in an oil-immiscible phase, oil-core particles can be formed in situ in or on an oil-immiscible phase by spraying a hydrophobic phase, including a propellant, into the bulk of, or onto the surface of, an oil-immiscible phase. In another embodiment, an oil-immiscible phase need not be present. Spraying of a hydrophobic phase, including a propellant, onto a solid surface results in a coating of intact oil-core particles of dimension and size distribution comparable to those produced by the suspension-based method described above.

In this embodiment, the hydrophobic phase contains a hydrophobic core material, a hydrophobic drug, an amphipathic surfactant, and a volatile propellant. A co-solvent can optionally, and in some embodiments, desirably be included. Each of the constituents listed can be, for example, any of the core materials, drugs, surfactants and solvents described above, without limitation. The propellant can be any suitable volatile liquid or gas, preferably those which spontaneously volatilize at atmospheric pressure and ambient temperature. These include, for example, fluorinated hydrocarbons such as 1,1,1,2,3,3,3-heptafluoropropane (HFA-227ea), and other examples of this class such as HFA-134a, or chiorofluorocarbons such as trichloromonofluoromethane, monochlorotrifluoromethane, dichloromonofluoromethane, monochlorodifluoromethane, trichlorotrifluoroethane, dichlorotetrafluoroethane, monochloropentafluoroethane, perfluorodimethylcyclobutane, dichlorodifluoromethane (CFC 12) and various freons. Supercritical fluids can also be used. Liquified carbon dioxide can be employed. In addition, other propellants can be included such as dimethyl ether. Preferred propellants are those that are relatively environmentally benign, for example, hydrofluorocarbons HFC-134a and HFC-227ea.

In embodiments in which oil-core particles are to be made pursuant to an aerosol delivery for immediate internal use, for example, the propellant must, of course, meet FDA approval. Further, the use of an approved metered dose inhaler (MDI) such as that disclosed in WO 96/32151 to Glaxo Wellcome Inc., for example, is generally desirable. In embodiments in which a propellant is used to make oil-core particles not for immediate administration, this constraint is not present. Propellants can be present in volumes of from about 5% to about 95% (based on the total volume of the hydrophobic phase).

The propellant can cause, or assist in, the dissolution of the hydrophobic phase components, but in some embodiments a co-solvent is desirable. The amount of co-solvent is chosen to produce a homogeneous solution of the hydrophobic core material, hydrophobic drug, and amphipathic surfactant. Any co-solvent mentioned herein can be considered suitable for use with a propellant. Some co-solvents will be present from about 2% to about 50% (by volume) of the hydrophobic phase. For example, ethanol can be present from about 2% to about 50%, for example, from about 5% to about 25%.

Propellants are to be introduced at pressures that allow convenient handling and to allow their use as liquids. Those of skill in the art will readily recognize appropriate pressures and handling techniques. Pressure-resistant vessels will generally be required for this method. After introduction of hydrophobic core material, hydrophobic drug, and amphipathic surfactant to such a vessel, an appropriate amount of propellant is introduced under pressure. Release of pressure through an actuator atomizes the contents of the flask and results in the formation of oil-core particles that are equivalent to those produced by suspension-based methods. Upon this atomization of the hydrophobic phase into droplets, and simultaneous release from pressure, the propellant volatilizes essentially instantaneously to form physiologically active oil-core particles, eliminating a solvent removal step separate from a particle formation step. Suitable actuators are commercially available, for example, from Precision Valve Corp. (Yonkers, N.Y.), or from Bespak, Inc. (Apex, N.C.). The Precision 21-85 Series of two-piece actuators provides examples of suitable actuators. Orifice sizes can range from about 0.005 inches to about 0.100 inches, preferably from about 0.008 to about 0.040 inches, more preferably from about 0.010 to about 0.025 inches. Mechanical breakup components can be included also.

### Post-Particle Formation Procedures

The products so produced can be sterilized by terminal sterilization, through methods such as that achieved with an autoclave or gamma irradiation, for example. Another method of sterilization useful for the inventive particles and suspensions thereof includes aseptic processing, using sterile filters to transfer liquid phases into sterile vessels. Such methods are known to those of skill in the art, and include the use of, for example, 0.2 μm PTFE filters for solvent-containing phases, and 0.2 μm nylon, polycarbonate, or cellulose acetate filters for aqueous phases.

Sterility testing of product lots is carried out directly after the lot is manufactured as a final product quality control test. Testing is done in accordance with various procedures found in the U.S. Pharmacopeia (U.S.P.) and FDA regulations.

Further optional manipulations of the invention particles include washing away of unincorporated drug, altering the drug or excipients, and adjusting concentration by concentrating or diluting the suspension.

### Characterization of the Pharmaceutical Compositions

In order to compare the suspensions, the following parameters are defined. The amount of the drug in the preparation is determined by an appropriate assay as described below. The yield of the drug is defined by the following equation.

$$Yield ( \% ) = \frac{Amount Recovered ( mg )}{Amount Input ( mg )} \cdot 100$$

In the above equation, the amount recovered is the amount of the drug determined to be in the suspension and the amount input is the total amount of the drug used in the preparation of the particles. The concentration of drug in the preparation is assayed (for example, by high pressure liquid chromatography, by enzyme-linked immunosorbent assay, by spectrophotometry, by bioassay, etc.), the total volume of the preparation is measured, and the amount of drug recovered is calculated as the product of concentration times volume.

For particles large enough to be separated by centrifugation, the ratio of the relative volume of the particulate fraction and the relative volume of the suspension is defined as the lipocrit. The suspension is centrifuged in hematocrit-type capillary tubes to produce a particulate fraction (which may either sink or float depending on the relative densities of particles and suspending medium) and a clarified fraction. Using the standard technique of hematocrit measurement, the relative volumes- of the particulate fraction and of the suspension are given by the distance from the one end of the particulate fraction to the other end of the particulate fraction, and from the bottom of the suspension after centrifugation to the top of the suspension, respectively. The lipocrit is given by the following equation.

lipocrit (%)=height of particle fraction/height of suspension×100

The concentration of unencapsulated drug in the suspending medium is determined (for particles large enough to be separated by centrifugation) by removing the particles from the suspending medium by centrifugation at 600-800×g for 10 minutes in a clinical centrifuge, or 7000×g for 3 minutes in a microfuge, isolating this suspending medium, and assaying the concentration of drug in the clarified suspending medium. This method has been found to give reliable results when the particles do not have crystallized drug present; the presence of crystals can be determined by, for example, microscopy.

The loading of drug is given by the following equation, assuming that the amount of unencapsulated drug is small (less than about 5% of the total in the suspension). Loading (mg/mL)=concentration of drug in suspension (mg/mL)/(lipocrit/100).

The inventive pharmaceutical preparations display a half time for drug release that is suitable for a wide range of applications. The half time is defined as the amount of time required for one half of the encapsulated drug to be released from the core of the oil-core particles. The half time for the inventive particles can be at least 10 hours, or at least about 20 hours, or at least about 30 hours. Different applications will have different optimum half times for drug release.

### Preparation and Usage of Pharmaceutical Compositions

The physiologically active oil-core particles of the invention form a part of pharmaceutical compositions which are to be administered to living beings for the diagnosis of, prophylaxis against, or treatment of an undesired condition, existing or threatened. Preferred pharmaceutical compositions include the inventive physiologically active particles, which can be suspended in an oil-immiscible medium such as water or aqueous solutions of sodium chloride, pharmaceutical excipients, and buffered solutions in the pH range of from about 2 to about 10. Preferred pharmaceutical excipients include phosphate, citrate, acetate, glucuronate, polysorbate, carboxymethylcellulose, gelatin, glucose, mannose, trehalose, mannitol, lysine, sorbitol, as well as amino acids, or salts, including alkali or alkali metal salts of the above excipients that can form a salt, as well as such salts of halides, citrate, pyrophosphate, or sorbate and lactate.

The pharmaceutical compositions can be formulated and stored in the form of □semi-solid dosage□forms, which means an aqueous suspension of the physiologically active oil-core particles of the invention. A semi-solid dosage form can be formed by addition of an aqueous medium to a solid dosage form of the particles of the invention, or can be formed directly by the methods disclosed herein. Thus, amorphous powders, tablets, capsules, aerosols, wafers, transdermal patches, suppositories, or implants can be formulated with the particles of the invention. Amorphous powders can be formed by lyophilization of a semi-solid dosage form of the particles of the invention. Tablets, capsules, aerosols, wafers, patches, suppositories, and implants can be formed by techniques well known to those in the art.

The pharmaceutical compositions of the invention can be administered to living beings parenterally by injection or by gradual infusion over time. The compositions can be administered intravenously, intraperitoneally, intramuscularly, subcutaneously, intracavity, or transdermally. The pharmaceutical compositions of the invention can also be administered enterally. The pharmaceutical compositions can be administered intraarticularly, epidurally, intrathecally, intralymphatically, orally, submucosally, transdermally, rectally, vaginally, intranasally, intraocularly and by implantation under various kinds of epithelia, including the bronchial epithelia, the gastrointestinal epithelia, the urogenital epithelia, and various mucous membranes of the body. Other methods of administration will be known to those skilled in the art.

For some applications, such as subcutaneous administration, the dose required can be quite small, but for other applications, such as intraperitoneal administration, the required dose can be very large. While doses outside the dosage range given below can be given, this range encompasses the breadth of use for practically all drugs. Generally, the dosage will vary with the age, condition, sex, and extent of the undesired condition in the patient, and can be determined by one skilled in the art. The dosage range appropriate for human use includes a range of from about 0.1 to 6,000 mg of the physiologically active substance per square meter of body surface area.

The methods of the invention are appropriate for use with physiologically active agents that would be sensitive to heating during the encapsulation process, and also allow aseptic processing by filtration without heating the solutions used in processing.

The invention will be further described in the following examples, which do not limit the scope of the invention described in the claims.

### EXAMPLES

The following examples illustrate the properties and performance of certain aspects of some embodiments of the physiologically active compositions of the invention.

### Example 1

### Preparation of a Pharmaceutical Composition

The pharmaceutical composition was prepared by a single emulsification process. The aqueous phase contained 5 mM lysine (Sigma Chemical Company, St. Louis, Mo.), and 5% sorbitol (J. T. Baker, Phillipsburg, N.J.) in HPLC grade water. The pH of the aqueous phase was approximately 10, in order to minimize the solubility of bupivacaine in the aqueous phase and keep the drug partitioned into the lipids. The surfactant stock solution contained 25 mM dipalmitoyl phosphatidylglycerol (DPPG), 100 mM dioleoyl phosphatidylcholine (DOPC), 125 mM cholesterol in chloroform. DPPG, and DOPC were from Avanti Polar Lipids (Alabaster, Ala.), and cholesterol and chloroform were from Spectrum Chemical Manufacturing Corp. (Gardena, Calif.). 73.9 mg of bupivacaine free -base was added to 2.2 mL of the surfactant stock solution. The bupivacaine free base was converted from bupivacaine hydrochloride that was purchased from Spectrum Chemical Manufacturing Corp. (Gardena, Calif.). 2.1 mL of tricaprylin (Avanti Polar Lipids, Alabaster, Ala.), and 0.65 mL of chloroform, were added to 2.2 mL of surfactant stock solution containing bupivacaine free-base. The surfactant stock solution containing tricaprylin and bupivacaine free base was added to 25 mL of the aqueous phase and mixed at 4000 rpm for 1 minute using a Homo mixer (Tokushu Kika Kogyo Co., Ltd., Osaka, Japan). This resulted in the formation of an oil-in-water emulsion. The emulsion was poured into 25 mL of aqueous solution and the solvent was evaporated using 75 scfm (standard cubic feet per minute flow rate) nitrogen for 30 minutes. After chloroform removal, the emulsion volume was adjusted to 50 mL with HPLC grade water to adjust the final concentration of lysine and sorbitol to 5 mM and 5%, respectively. The particles were washed twice by centrifuging at 800×g for 10 minutes to separate the unencapsulated drug from encapsulated drug (which floats) and allow a determination of yield in the encapsulated fraction. After centrifugation, the infranatant was removed and the particles were suspended in 5 mM lysine/5% sorbitol in HPLC grade water.

In this and all examples involving bupivacaine (except where noted; Example 6), the concentration of bupivacaine in the pharmaceutical composition, and the infranatant was determined by isocratic reverse phase high pressure liquid chromatography (Hewlett-Packard, Wilmington, Del.) using a C18 column (Waters), 80% 10 mM KH₂PO₄ (pH 2.1) and 20% acetonitrile as the mobile phase, a flow rate of 1 mL/min and 205 nm for the wavelength of detection. In this and other examples, the mean particle diameter was determined on a laser scattering particle size distribution analyzer (Model LA-910, Horiba Instruments, Irvine, Calif.) using the volume-weighted distribution base and a relative refractive index of 1.10-0.00i. The mean particle diameter, yield of the drug and drug loading are reported in Table 1.

As shown in Table 1, the median particle diameter is 16.0 microns, and the particle size distribution is 8.2 microns. Further, the yield (93%) of this process was excellent.

### Example 2

### Preparation of a Pharmaceutical Composition without Solvent

A comparative example was carried out to compare particles prepared without volatile solvent to those prepared using volatile solvent (Example 1).

The pharmaceutical composition was prepared by a single emulsification process. The aqueous phase was as in example 1. The lipid stock solution contained 26 mM dipalnitoyl phosphatidylglycerol (DPPG), 105 mM dioleoyl phosphatidylcholine (DOPC), 131 mM cholesterol in 2.1 mL of tricaprylin. Tricaprylin was from Avanti Polar Lipids (Alabaster, Ala.). The mixture of lipids and tricaprylin was heated at 50□ C for 2 hours. 73.9 mg of bupivacaine free-base was added to the lipid mixture and it was heated for 1 hour at 50° C. The lipid mixture containing the bupivacaine free base was added to 25 mL of the aqueous phase. The lipids and aqueous were mixed at 4000 rpm for 1 minute using a Homo mixer (Tokushu Kika Kogyo Co., Ltd., Osaka, Japan). This resulted in the formation of an oil-in water emulsion. The emulsion was poured into 25 mL of aqueous solution and washed twice by centrifuging at 800×g for 10 minutes. After centrifugation, the infranatant was removed and the particles were suspended in 5 mM lysine/5% sorbitol in HPLC grade water.

The median particle diameter, yield of drug and drug loading are summarized in Table 1. The process carried out without volatile organic solvent had a particle size distribution that was measurably poorer than that of Example 1, and the yield (74%) was similarly poor.

### Example 3

### Preparation of a Pharmaceutical Composition with Solvent Solvent Removed Before Emulsifying

A comparative example was carried out to compare particles prepared with volatile solvent that was removed before the addition of an aqueous phase, to those prepared with volatile solvent that was removed after the addition of an aqueous phase.

The pharmaceutical composition was prepared by a single emulsification process. The aqueous phase and surfactant stock solution were as in Example 1. 73.9 mg of bupivacaine free-base was added to 2.2 mL of the surfactant stock solution. 2.1 mL of tricaprylin (Avanti Polar Lipids, Alabaster, Ala.), was added to the 2.2 niL of surfactant stock solution containing bupivacaine free base. The chloroform was evaporated from the solvent phase containing tricaprylin and bupivacaine free-base, using 10 scfm nitrogen for 3 hours. Evaporation of the chloroform from the lipids was confirmed by the increase of viscosity and lack of clarity in the mixture.

The lipids were added to 25 mL of the aqueous phase. The lipids and aqueous phase were mixed at 4000 rpm for 1 minute using a Homo mixer (Tokushu Kika Kogyo Co., Ltd., Osaka, Japan). This resulted in the formation of an oil-in water emulsion. The emulsion was poured into 25 mL of aqueous solution and washed twice by centrifuging at 800×g for 10 minutes. After centrifugation, the infranatant was removed and the particles were suspended in 5 mM lysine/5% sorbitol in HPLC grade water.

The median particle diameter, yield of drug and drug loading are summarized in Table 1. The particle size distribution is quite broad, and the yield is also inferior to these same properties of the particles of Example 1.

### Example 4

### Preparation of a Pharmaceutical Composition by Extrusion

A comparative example was carried out to compare particles prepared by an extrusion method to those prepared by an emulsification method.

The pharmaceutical composition was prepared by a single emulsification process. The aqueous phase and surfactant stock solution were as in Example 1. 73.9 mg of bupivacaine free-base was added to 2.2 mL of the surfactant stock solution. The bupivacaine free base was converted from bupivacaine hydrochloride that was purchased from Spectrum Chemical Manufacturing Corp. (Gardena, Calif.). 2.1 mL of tricaprylin (Avanti Polar Lipids, Alabaster, Ala.) and 0.65 mL chloroform were added to 2.2 mL of surfactant stock solution containing bupivacaine free base. The surfactant stock solution containing tricaprylin and bupivacaine free base was extruded through a 21-gauge needle attached to a 10 cc glass syringe, at a rate of 5 mL per 2.15 minutes into 50 mL of the aqueous phase. The aqueous phase was heated to 45° C. and gently stirred. The solvent was evaporated using 70 scfm nitrogen for 30 minutes. After chloroform removal, the suspension volume was adjusted to 50 mL with HPLC grade water to adjust the final concentration of lysine and sorbitol to 5 mM and 5%, respectively. The particles were washed twice by centrifuging at 800×g for 10 minutes. After centrifugation, the infranatant was removed and the particles were suspended in 5 mM lysine/5% sorbitol in HPLC grade water.

The median particle diameter, yield of drug and drug loading are summarized in Table 1. The particle size distribution was not comparable to that of the particles of Example 1, and the yield was also inferior to the method of Example 1.

TABLE 1
Characteristics of Lipid-Containing Compositions with and
without Solvent.

| | Median Particle | | | |
|---|---|---|---|---|
| | Diameter | Chloroform | Yield | Loading |
| Example | (μm ± SD) | Present | (%) | (mg/mL) |
| 1 | 16.0 ± 8.2 | yes | 93 | 27.3 |
| 2 | 19.9 ± 24.9 | no | 74 | 29.8 |
| 3 | 15.5 ± 32.1 | yes | 83 | 35.4 |
| 4 | 29.4 ± 43.2 | yes | 87 | 30.4 |

### Example 5

### Preparation of a Pharmaceutical Composition with Paclitaxel, Using Tributyrin

The pharmaceutical composition was prepared by a single emulsification process. The aqueous phase contained 5% glucose (McGaw, Irvine, Calif.), and 5 mM lysine (Sigma Chemical Company, St. Louis, Mo.) in HPLC grade water. The surfactant stock was as in Example 1. 25 mg of paclitaxel (Aldrich Chemical Company, Milwaukee, Wis.) was added to 2.16 mL of the surfactant stock solution. 2.0 g of tributylin (Sigma Chemical Company, St. Louis, Mo.) and 0.61 mL of chloroform was added to 2.16 mL of surfactant stock solution containing paclitaxel. The surfactant stock solution containing tributyrin and paclitaxel was added to 20 mL of the aqueous phase and mixed at 4000 rpm for 1 minute using a Homo mixer (Tokushu Kika Kogyo Co., Ltd., Osaka, Japan). This resulted in the formation of an oil-in-water emulsion. The emulsion was poured into 30 mL of aqueous solution and the solvent was evaporated using 70 scfm nitrogen for 30 minutes. After chloroform removal, the suspension volume was adjusted to 50 mL with HPLC grade water to adjust the final concentration of glucose and lysinc to 5 mM and 5%, respectively. The particles were washed twice by centrifuging at 800×g for 10 minutes. After centrifugation, the supernatant was removed and the particles were suspended in 5% glucose in HPLC grade water.

The concentration of paclitaxel in the pharmaceutical composition and the infranatant was determined by isocratic reverse phase high pressure liquid chromatography (Hewlett-Packard, Wilmington, Del.) using a Primesphere 5 C18 column (Phenomenex), 65% acetonitrile and 35% HPLC grade water as the mobile phase, a flow rate of 1 mL/min and 230 nm for the wavelength of detection. The median particle diameter, yield of drug and drug loading are summarized in Table 2.

TABLE 2
Characteristics of Lipid-Containing Compositions

| | Median Particle | | |
|---|---|---|---|
| | Diameter | | Loading |
| Example | (μm, ± SD) | Yield (%) | (mg/mL) |
| 5 | 16.3 ± 6.5 | 78.5 | 8.4 |

### Example 6

### Characteristics of Liquid Oil and Solid Oil-Core Pharmaceutical Compositions

The pharmaceutical composition was prepared by a single emulsification process. The aqueous phase contained 5 mM lysine (Sigma Chemical Company, St. Louis, Mo.), and in some cases 4% polyvinyl alcohol (Sigma Chemical Company, St. Louis, Mo.) in HPLC grade water. The surfactant stock solution was as in Example 1. The solvent phase consisted of varying amounts of surfactant stock solution and chloroform with a constant mass of either triolein (liquid at room temperature) or tristearin (solid at room temperature). Triolein was from Avanti Polar Lipids (Alabaster, Ala.), and tristearin was from Sigma Chemical Company (St. Louis, Mo.). Bupivacaine free-base and phospholipid (PL) were added to the solvent phase at various concentrations. Tristearin did not completely dissolve at room temperature in this volume of chloroform, so the hydrophobic phase was dissolved at 37° C. then quickly brought to room temperature and mixed with the aqueous phase while still clear. The solvent phase containing surfactant mix and either triolein or tristearin and bupivacaine free base was added to 20 mL of the aqueous phase and mixed at 4000 rpm for 60 seconds using a Homo mixer (Tokushu Kika Kogyo Co., Ltd., Osaka, Japan). This resulted in the formation of an oil-in-water emulsion. The emulsion was poured into 30 mL of aqueous solution and the chloroform was evaporated using 50 scfm nitrogen for 20 minutes. After chloroform removal, the suspension volume was adjusted to 50 mL with HPLC grade water to adjust the final concentration to 5 mM lysine. The particles were washed twice by centrifuging at 600 x g for 10 minutes. After centrifugation, the infranatant was removed and the particles were suspended in 5 mM lysine in HPLC grade water.

The determination of bupivacaine was carried out as in Example 1, except that 80% 170 mM KH₂PO₄ (pH 2.5) was used instead of 10 mM KH₂PO₄. The yield of the drug, drug loading and particle diameters are summarized in Table 3. The mean particle diameters are listed in Table 3.

TABLE 3
Characteristics of Oil-Core Particle Composition with Liquid Oil and Solid Oil.

| | | | | | | Total | | Major | | |
|---|---|---|---|---|---|---|---|---|---|---|
| | | | Mass of | Mass of | Mass of | Lipid | | Peak | | |
| Lipids | Aqueous | Mass of | PC | Chol | TG | Phase | Drug | Diameter | Yield | Loading |
| (PL) | Phase | PL (mg) | (mg) | (mg) | (grams) | (mL) | (mg) | (μm) | (%) | (mg/mL) |
| Triolein | 5 mM Lysine | 38.35 | 161.92 | 97.67 | 2.00 | 4.36 | 150 | 34.25 | 75 | 39.8 |
| Triolein | 5 mM Lysine | 38.35 | 161.92 | 99.60 | 2.00 | 4.44 | 200 | 22.80 | 76 | 59.3 |
| Triolein | 5 mM Lysine | 94.22 | 397.72 | 244.65 | 2.00 | 7.39 | 150 | 22.80 | 74 | 26.7 |
| Triolein | 4% PVA/ | 38.35 | 161.92 | 99.60 | 2.00 | 4.39 | 150 | 17.38 | 59 | 54.0 |
| | 5 mM Lysine | | | | | | | | | |
| Tristearin | 4% PVA/ | 38.35 | 161.92 | 99.60 | 2.00 | 9.89 | 150 | 11.56 | 15 | 4.0 |
| | 5 mM Lysine | | | | | | | | | |
| Tristearin | 4% PVA/ | 38.35 | 161.92 | 99.60 | 2.00 | 9.94 | 200 | 11.56 | 17 | 7.6 |
| | 5 mM Lysine | | | | | | | | | |
| Tristearin | 4% PVA/ | 95.71 | 404.00 | 248.52 | 2.00 | 9.96 | 150 | 13.25 | 12 | 2.3 |
| | 5 mM Lysine | | | | | | | | | |

### Example 7

### Characteristics of Lipid-Core Compositions Made with Simple Saturated Triglycerides of Various Acyl Chain Lengths and Physical Forms

An example was carried out to compare particles made with different triglycerides. The physical properties of these core materials are given in Table 4. Values in Table 4 are from Small, D. M., “The Physical Chemistry of Lipids,” New York: Plenum Press, 1986, and from Weast, R. C. (ed.) “CRC Handbook of Chemistry and Physics, 55^{th} ed.,” Cleveland: CRC Press, 1974. Entries in Table 4 labeled “nr” were not reported in these references.

TABLE 4
Physical Data of Triglycerides (TG)

| Batches as in | | chain | Tm, α | Tm, β′ | Tm, β | density | density |
|---|---|---|---|---|---|---|---|
| Table 5 | TG | length | (° C.) | (° C.) | (° C.) | (β form) | (liquid) |
| A,I | Tributyrin | C4 | nr | nr | −75 | nr | 1.034 |
| B,J | Tricaproin | C6 | nr | nr | −25 | nr | 0.987 |
| C,K | Tricaprylin | C8 | nr | −21 | 8.3 | nr | 0.954 |
| D,L | Tricaprin | C10 | −15 | 18 | 31.5 | nr | 0.891 |
| E,M | Trilaurin | C12 | 15 | 35 | 46.5 | 1.057 | 0.880 |
| F,N | Trimyristin | C14 | 33 | 46.5 | 57 | 1.050 | 0.872 |
| G,O | Tripalmitin | C16 | 44.7 | 56.6 | 66.4 | 1.047 | 0.875 |
| H,P | Tristearin | C18 | 54.9 | 64 | 73.1 | 1.043 | 0.863 |

The aqueous phase was 5 mM lysine, with or without 4% by weight polyvinyl alcohol (PVA) (Sigma, 30K-70K MW). The hydrophobic phase contained 2.0 g of the triglyceride (TG) indicated in Table 4 (obtained from Sigma and from Nu-Chek-Prep, Inc., Elysian, Minn.), 40.2 mg sodium DPPG, 170 mg DOPC, 46.1 mg cholesterol, and 100 mg bupivacaine free-base; this was brought to 10 ml with chloroform. Tristearin did not completely dissolve at room temperature in this volume of chloroform, so the hydrophobic phases for batches H and P were dissolved at 37° C. then quickly brought to room temperature and mixed with the aqueous phase while still clear. The hydrophobic phase was emulsified with 20 mL of aqueous phase in a TK Homo mixer at either 4000 rpm for 60 seconds (batches A-H), or 2000 rpm for 30 seconds (batches I-P), to form an oil-in-water emulsion. (Mixing speed and time were reduced in the presence of PVA to give roughly comparable particle sizes.) The aqueous phase for batches A-H was 5 mM in lysine, and for batches I-P was 5 mM in lysine and 4% polyvinyl alcohol (PVA) by weight. The emulsions were diluted into 30 ml aqueous phase, and the chloroform was evaporated by flushing the surface of the suspension with nitrogen. The suspensions were brought back to 50 mL by the addition of water (to bring the lysine concentration to approx. 5 mM), then preparations containing PVA were further diluted by addition of 50 mL 5 mM lysine. The suspensions were washed by centrifugation at 600×g for 10 minutes. Any floating fractions or pellets were separated from the remainder, diluted with aqueous phase and resuspended if possible (this was not possible for floating fractions from preparations E and F, which formed tightly packed cakes under these conditions), then centrifuged again and isolated for analysis. Observations of particle density with respect to the suspending medium, the median particle diameter, yield of drug, approximate pellet volume and, loading of drug, and % of theoretical maximum drug loading are given in Table 5. Theoretical maximum drug loading is approximated by the initial amount of drug used, divided by the volume of oil used. Entries labeled “n.d.” were not determined, because the particles made could not be resuspended following the procedure. Batch F produced two fractions, fr. 1 which was the major fraction by volume, and fr. 2, a minor fraction.

Those fractions that exhibit pelleting may do so because their contents are denser than the suspending medium. Alternatively, since bupivacaine crystals are denser than water, fractions that pellet may include precipitated bupivacaine free-base which is not truly encapsulated, and this may exaggerate the reported yield. Fractions that float do not contain bupivacaine crystals at the time of separation, but rather contain solubilized drug. The low yield for batch J after washing may be due to the small difference in density between particles and the suspending medium, together with the viscosity of the PVA-containing suspending medium. The loading for batch J is similar to that for batch K, implying that tricaproin particles containing large amounts of drug can be produced, although not isolated quantitatively with the technique used here. Filtration, for example, can be used to collect particles that have densities similar to that of the suspending medium.

The triglycerides in batches A through C and I through K are presumed to be in a liquid state at room temperature (that is, above the Tm for the most stable form, the beta form), while the triglycerides in preparations F through H and N through P are presumed to be in a solid state (at some time after removal of the solvent (that is, below the Tₘ for the alpha form). The fact that suspensions made with tricaprin and trilaurin float in 4% PVA/ 5 mM lysine (density measured to be approx. 1.007 at room temperature) and in 5 mM lysine suggests that the lipid in these suspensions is most likely in the liquid form. Yields for batches L and M are as high as those for batches I through K, and much higher than yields for batches N through P. Thus particles containing solid triglyceride appear not to be capable of high loading with this method, while particles containing triglycerides in other forms may be prepared with high drug loading by this method.

TABLE 5
Characteristics of Oil-Core Particle Compositions with Various Triglycerides

| | | particle density | | | | |
|---|---|---|---|---|---|---|
| | | (w/r/t aq. | median diameter | yield | approx. pellet | loading |
| Batch | TG | phase) | (microns) | (%) | vol. (mL) | (mg/ml) |
| A | tributyrin | pellets | 12.3 | 100% | 3.02 | 33 |
| B | tricaproin | floats | 15.1 | 79% | 3.41 | 23 |
| C | tricaprylin | floats | 12.9 | 84% | 3.17 | 27 |
| D | tricaprin | floats | 13.2 | 93% | 3.19 | 29 |
| E | trilaurin | floats | could not be | n.d. | n.d. | n.d. |
| | | | resuspended | | | |
| F (fr. 1) | trimyristin | pellets | 23.6 | 4% | 4.95 | 0.7 |
| F (fr. 2) | trimyristin | floats | could not be | n.d. | n.d. | n.d. |
| | | | resuspended | | | |
| G | tripalmitin | pellets | 23.9 | 42% | 8.26 | 5 |
| H | tristearin | | none: formed a | n.d. | n.d. | n.d. |
| | | | water-in-oil emulsion | | | |
| I | tributyrin | pellets | 10.9 | 79% | 3.12 | 25 |
| J | tricaproin | floats | 15.4 | 17% | 0.85 | 20 |
| K | tricaprylin | floats | 11.9 | 62% | 2.77 | 22 |
| L | tricaprin | floats | 11.6 | 68% | 2.83 | 24 |
| M | trilaurin | floats | 11.3 | 58% | 2.18 | 26 |
| N | trimyristin | pellets | 16.8 | 2% | 3.95 | 0.5 |
| O | tripalmitin | pellets | 11.9 | 3% | n.d. | n.d. |
| P | tristearin | pellets | 13.3 | 11% | n.d. | n.d. |

### Example 8

### Efficacy of Bupivacaine-Containing Pharmaceutical Preparations

A triolein-core particle suspension was made by combining 2 g triolein, 104 mg bupivacaine free base, 0.61 mL chloroform, and 2.1 mL of a chloroform solution of 100 mM DOPC/25 mM DPPG 125 mM cholesterol. This hydrophobic phase was added to 25 mL of 5% sorbitol/10 mM lysine, and emulsified 60 seconds at 4000 rpm in a TK Homo mixer to form an oil-in-water emulsion. Solvent was removed and particles washed as described in Example 7. Three such batches were combined, and the concentration adjusted to 15 mg bupivacaine free base/mL.

A tricaprylin-core particle suspension was made as above, substituting tricaprylin for triolein. Of the original bupivacaine supplied for these two preparations, 83 to 85% was recovered in fractions that float upon centrifugation, thus was incorporated into lipid-core particles. Triolein-core particles after washing were 17.8+/−6.6 microns diameter (volume weighted, measured with a Horiba LA-910 light scattering particle analyzer, using relative refractive index 1.10-0I), with a lipocrit of 53%. Tricaprylin-core particles were 15.1+/−6.2 microns diameter, with a lipocrit of 48%.

Efficacy was investigated in a rat sciatic nerve block model, using a thermal paw stimulator to quantitate sensory block, in a method drawn from J. Curley et al., “Anesthesiology,” 84, 1401-1410 (1996). Heat from a high-intensity lamp was focused through a glass plate onto the plantar paw surface. The time until the rat lifted its foot was noted. The maximum time of exposure to the stimulus was 20.5 seconds. A baseline (time zero) response was determined for both hind legs, rats were lights anesthetized with Halothane, then the left leg of each rat was injected at the sciatic nerve with 200 microliters of test material. The right leg served as an uninjected control; response was tested on both legs at various times post-injection. Motor block was scored by noting the “clubbing” (curling up) of the affected foot. Full clubbing, partial clubbing, and no clubbing were scored as 2, 1, and 0 respectively, and scores at each time point were averaged for all rats in a group. A 200 microliter aliquot of either 1.5% bupivacaine free base (total 3.0 mg) in a lipid-core particle suspension or 0.56% bupivacaine phosphate solution (equivalent to 0.5% bupivacaine-HCl monohydrate or 0.82 mg bupivacaine free base) was injected at the sciatic nerve in the left leg of each rat (lightly anesthetized with Halothane). On one day, one group of three rats was used for each preparation. The study was repeated with fresh rats on the following day, for a total of two groups of three rats each per preparation.

In a previous experiment with similar formulations of triolein and tricaprylin, it had been determined that bupivacaine did not crystallize out within 7 days after preparation of the particles, stored at either room temperature of 4° C. For the experiment described here, particles were injected two days after emulsification, and during these two days the preparations were kept at room temperature. On the first day, one group of three rats was used for each preparation. On the following day the study was repeated with fresh rats, for a total of six rats per preparation. Three preparations were compared: 1.5% bupivacaine free base (total 3.0 mg in 200 microliters) in a triolein-core suspension, 1.5% bupivacaine free base in a tricaprylin-core suspension, and 0.56% bupivacaine phosphate solution (equivalent to 0.82 mg bupivacaine free base in 200 microliters).

The harvested particles were spherical by light microscopy, and had diameters of 18.2 6.5 microns diameter (volume weighted mean SD, by light scattering). By a lipocrit assay, upon centrifugation, floating particles occupied 70% of the volume of the suspension. The concentration of bupivacaine free-base in the suspension was 15 mg/mL (equivalent to 1.8% bupivacaine HCl monohydrate). The washed floating fraction contained 83% of the bupivacaine originally supplied.

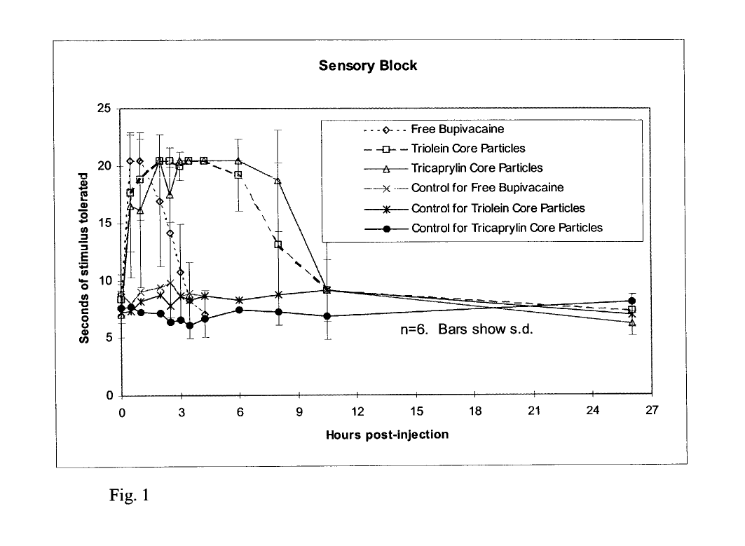

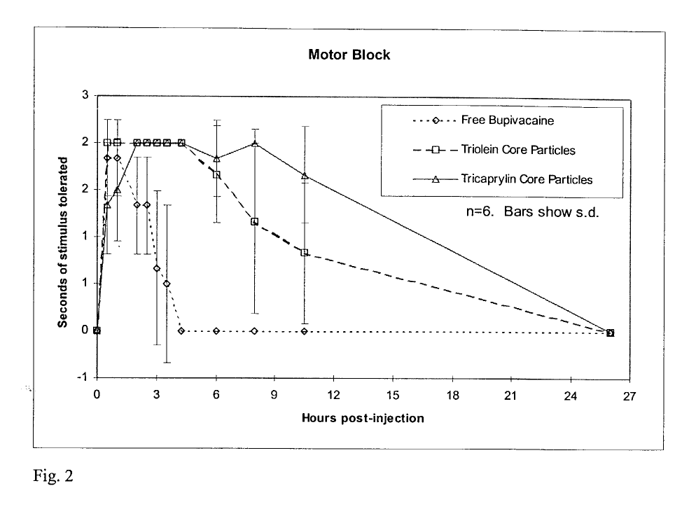

No evidence of discomfort or irritation was noted upon deposition of the alkaline lipid-core particle suspension in proximity to the sciatic nerve. As shown in FIGS. 1 and 2, the duration of sensory- and motor-block obtained with the bupivacaine lipid-core particle suspension was at least double that obtained with the free (unencapsulated) drug solution.

The results show that sustained release of drugs can be achieved using oil-core particles made from unsaturated triglycerides made with the methods of the invention.

### Example 9

### Solubility of Drug Increased with Triglycerides of Shorter Acyl Chain Length

On the day following the experiment described in Example 8, remaining material not used for the experiment was used to test stability. A portion of each suspension was stored at room temperature, and another portion stored in a refrigerator at approximately 6° C. A month after emulsification, the triolein suspensions stored at both temperatures, which upon storage had risen to the tops of their container, were noted to be difficult to resuspend by gentle shaking, so all suspensions were examined by microscopy. Long threadlike crystals, presumed to be of bupivacaine free-base since they dissolve upon lowering the pH of the preparation, were seen in the triolein-core suspensions stored at both temperatures. No such crystals were seen in the tricaprylin-core suspension.

To confirm that the formation of bupivacaine crystals was related to differing solubility of the drug in the oils used, a solubility experiment was performed. Varying amounts of bupivacaine free-base were added to 1.0 g aliquots of either triolein or tricaprylin in small vials with Teflon-lined screw caps. These vials were heated to ˜70° C., shaken vigorously, cooled to room temperature, then 1.0 mL of 10 mM lysine/5% sorbitol was added to each vial. The vials were put in a 4° C. cold room for a month. At the end of this time when inspected by eye, precipitates or large masses, apparently crystals of bupivacaine, were seen in vials containing 30 mg or more bupivacaine per g triolein, but not in vials containing 20 mg or less. For the tricaprylin samples, precipitates or large masses were seen in vials containing 50 mg or more bupivacaine per g tricaprylin, but not in vials containing 20, 30 or 40 mg bupivacaine per g tricaprylin. The vial containing 0 mg bupivacaine per g tricaprylin contained crystals, but these melted upon warming to room temperature, indicating that some of the tricaprylin in this vial had solidified. This vial was the only one in which a precipitate disappeared upon warming to room temperature. Thus bupivacaine free-base is more soluble in tricaprylin than in triolein.

### Example 10

### Paclitaxel in Triolein- and Tricaprylin-Core Particles

The procedure of Example 5 was used, substituting either triolein or tricaprylin for tributyrin, and isolating the washed particles as a floating fraction rather than as a pellet. The particles were examined by light microscopy. Crystals were observed within lipid droplets for both formulations. In contrast, such crystals were not observed in the tributyrin-core preparations of Example 5.

### Example 11

### Efficacy of Bupivacaine-Containing Pharmaceutical Preparations

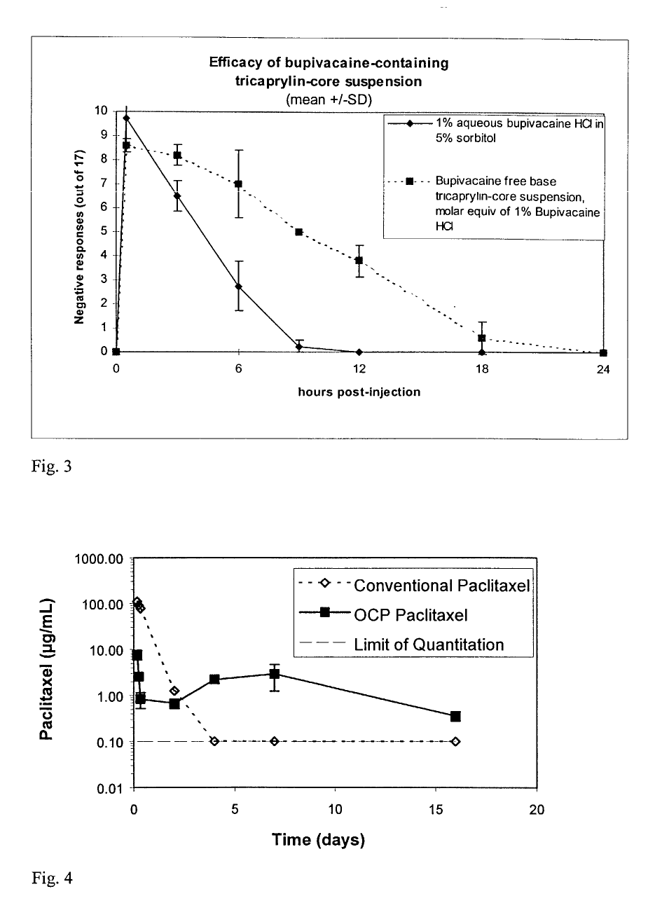

A suspension of bupivacaine free base in tricaprylin-core particles was made as described in Example 8, and adjusted to 0.84% Bupivacaine free base, equivalent to 1% bupivacaine HCl monohydrate. This is used in an assay of anesthetic effect. Previously shaved guinea pigs (4-5 per group) were marked with a stencil in the form of a circular array, with 17 tick marks at various radii and directions from the center of the array. Animals anesthetized with Halothane were injected intracutaneously with 1.0 mL of either the tricaprylin-core bupivacaine suspension or a solution of 1% bupivacaine HCl monohydrate in 5% sorbitol. The response to pin pricks was tested at various post-injection intervals (30 min, 3, 6, 9, 12, 18, 24, 30 and 36 hours) and the results summarized in FIG. 3. A vocalization or muscle twitch was considered a positive response. The guinea pigs' response to the bupivacaine solution decayed to half of its maximum at about 3.6 hours after injection. An equivalent (on a molar basis) dose of bupivacaine free base tricaprylin-core suspension took about 8½ hours to decay to the same number of negative responses, and took about 11 hours to decay to half of the maximal response for this formulation.

### Example 12

### In Vivo Concentration Time Course for Oil-Core Pharmaceutical Compositions

The in vivo concentration-time course for a conventional formulation of paclitaxel was compared to a formulation of oil-core particles containing paclitaxel (OCP Paclitaxel). OCP paclitaxel were prepared as described in Example 5, with the exception that 5 mM lysine was omitted from the aqueous phase. The particle diameters, yield, and loading were found to be, within experimental error, identical to those listed in Table 2 of Example 5.

“Conventional” formulations of paclitaxel were prepared by adding 6 mg paclitaxel to 1 mL of a mixture (1:1, v/v) of anhydrous alcohol and Cremophor® EL (Sigma Chemical Company, St. Louis, Mo.). Stocks of OCP paclitaxel and conventional paclitaxel were diluted in 5% glucose or sterile saline, respectively, to obtain 0.8 mg/mL injectable solutions. For each of the 8 time (4, 6, 8, 24, and 48 hours, 4, 7, and 16 days), 4 normal rats (male Sprague-Dawley; Harlan, Indianapolis, Ind.) were administered 16 mg/kg paclitaxel by injecting 5 mL of OCP paclitaxel or conventional paclitaxel by the intraperitoneal route. At the indicated time points, animals were euthanized and blood, peritoneal fluid, liver, and spleen samples were collected, processed and quickly frozen.

Paclitaxel concentrations in plasma, peritoneal fluid, and homogenized tissues were determined by HPLC after liquid-liquid extraction of the samples with diethyl ether followed by solid-phase extraction (Bond Elut; Varian, Harbor City, Calif.). The samples were then suspended in 45:15:40 acetonitrile:methanol:water, and 50 μL was injected onto a Waters Symmetry C18 column (Waters, Taunton, Mass.) with a Hewlett-Packard Model 1100 solvent delivery system (Hewlett-Packard, Wilmington, Del.). The mobile phase was a mixture of acetonitrile, methanol, and 0.2 M ammonium acetate at pH 5 (45:15:40) with a rate of 1 mL/min; detection was at a wavelength of 230 nm. Apparent terminal half-lives (T_{1/2beta}) were estimated from the terminal log-linear decline of the concentration-time profiles.

The results from peritoneal fluid analysis illustrate the most significant findings in this study and are presented in FIG. 4 and Table 6. Values in Table 6 are given in μg/mL for peritoneal fluid, and μg/g for liver and spleen tissues. Values in parentheses are standard deviation values. Number of samples (n) is four for each determination. Paclitaxel levels in plasma are not reported, since they were below the limit of detection by the HPLC method employed. The one-day entry for peritoneal fluid (entry marked “*” in Table 6) was not collected. FIG. 4 is a concentration-time course for paclitaxel in rat peritoneal fluid determined after a 16 mg/kg intraperitoneal bolus. In FIG. 4 the error bars indicate standard deviation, and four samples were recorded for each time point.

The concentration-time profile for paclitaxel in peritoneal fluid above shows a monoexponential decline for conventional paclitaxel with a half-life of 0.28 days. After conventional paclitaxel administration, no detectable drug concentrations were found 48 hours after treatment. In contrast, OCP paclitaxel shows a biphasic decline for paclitaxel concentrations in peritoneal fluid. The initial phase has a half-life of approximately 0.06 days while the terminal phase has a half-life of 4.23±0.99 days. After paclitaxel OCP dosing, persistent and significant drug concentrations were observed in peritoneal fluid for the following 16 days (through the endpoint of the study).

The concentrations sustained in peritoneal fluid after OCP paclitaxel injection were significantly larger than the minimum inhibitory concentration (0.085 ug/mL) required to induce microtubule bundling and other pertinent cytotoxic effects in vitro (E. K. Rowinsky et al., Cancer Research 48: 4093-4100, 1988). Further, the concentrations sustained after OCP paclitaxel injection were in the clinically effective range. As an indication of clinically therapeutic concentrations, the peak concentrations observed in the plasma of patients treated with recommended intravenous doses of conventional paclitaxel were 0.2 to 3.6 μg/mL (Physician's Desk Reference 54th Edition, 2000, Medical Economics Company, Montvale, N.J.). The inventive composition was able to sustain comparable concentrations in peritoneal fluid for at least 16 days. (This was not merely an effect of the intraperitoneal route of administration, since the conventional paclitaxel formulation administered intraperitoneally resulted in detectable concentrations for only two days.) This is a significant finding for a cell-cycle specific agent such as paclitaxel, where the duration of exposure is vital to produce maximal benefit from treatment. Thus, this study shows the superiority of the inventive composition.

TABLE 6
Mean Concentrations of Paclitaxel after Intraperitoneal Administration

| | |
|---|---|
| | concentration in Peritoneal Fluid, |
| Time (days) after | μg/mL (S.D.) |

| administration | Conv. Paclitaxe | OCP Paclitaxel |
|---|---|---|
| 0.17 | 111.11 (16.97) | 7.74 (1.76) |
| 0.25 | 90.88 (11.14) | 2.59 (0.02) |
| 0.33 | 77.89 (14.76) | 0.84 (0.32) |
| 1.00 | * | * |
| 2.00 | 1.26 (0.14) | 0.68 (0.16) |
| 4.00 | 0.00 (0.00) | 2.21 (0.40) |
| 7.00 | 0.00 (0.00) | 2.96 (1.71) |
| 16.00 | 0.00 (0.00) | 0.36 (0.08) |

### Example 13

### Preparation of Oil-Core Particles by Propellant-Based Method

In a pressure-resistant glass vial to which an aerosol valve and nozzle could be attached and sealed, 6.5 mg beclomethasone dipropionate (BDP), 0.812 mL tributyrin, 0.644 mL ethanol, and 48.7 mg dioleoyl phosphatidylcholine (DOPC) were placed. The valve to the glass vial was sealed, and 5.0 mL liquid hydrofluoroalkane 227ea (HFA-227ea) was introduced through the valve, under pressure. A Precision Valve Corp. Series 21-85, two-piece actuator with mechanical breakup (orifice size 0.020″ or 0.013″) was then pressed onto the valve stem. The container was swirled gently by hand until the solution appeared clear, indicating that the components had dissolved. The contents of the pressurized glass vial remained visually clear, with no turbidity or precipitate for greater than a month at room temperature, suggesting that the components remained in solution for at least this long.

The preparation was sprayed out of the actuator onto a microscope slide about 12 inches away from the nozzle, and inspected (dry, with no cover slip and no mounting medium) in a microscope, using a 40× objective. A calibrated micrometer scale was incorporated into the microscope ocular. The majority of refractile particles were from about 20 to about 25 microns in diameter. The formation of crystals was not noted.

For particle size distribution measurement in a Horiba LA-910 laser diffraction particle sizer, the same preparation was sprayed from the actuator onto the surface of a 0.9% sodium chloride solution in a beaker. The sodium chloride solution was swirled in the beaker as the preparation was being sprayed. The amount of preparation sprayed into the saline was sufficient to be usable (that is, in the appropriate turbidity range as indicated by the instrument). Using a relative refractive index of 1.10-0i and volume weighting, the output scan showed a minor peak of ˜600 microns diameter, accounting for about 3% of the total volume of particles, and a major peak of about 22 microns diameter. A drop of suspension was placed on a microscope slide, covered with a cover slip, and inspected in a microscope. The majority of particles were from about 20 to about 25 microns in diameter, and crystal formation was not noted, although it appeared that some surface material was being sloughed off of some of the particles to form nonrefractile vesicular structures.

### Example 14

### Substitution of Surfactant in Propellant-Based Method

An aerosol preparation as described in Example 13 was made by substituting dipalmitoyl phosphatidylcholine (DPPC) for DOPC. The pressurized solution again appeared clear in the glass container. When sprayed into the saline solution and the particle size distribution analyzed by the Horiba sizer, it showed a minor peak of ˜600 microns diameter and a major peak of about 18 microns diameter. By microscopy, this preparation (sprayed on a microscope slide or into saline) appeared similar to that described in Example 13.

### Example 15

### Solubilization of Drug in Propellant

Beclomethasone dipropionate (BDP) was soluble in neat tributyrin up to BDP concentrations slightly greater than 10 mg/mL of tributyrin. At approximately 8 mg BDP per mL of tributyrin, BDP was expected to be soluble in the mixture of oil and phospholipid after evaporation of the propellant and co-solvent. I-lowever, when ethanol was omitted from the mixture of Example 13, the resulting mixture (with propellant, in the vial, pressurized) was turbid. When ethanol was present in an amount of 10% of the total mixture volume, the preparation was clear, but when ethanol was present in an amount of only 5% (by volume), the preparation was turbid. Thus, ethanol can act as a co-solvent in the presence of HFA 227ea.

Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. Physiologically active oil-core particles, wherein each particle comprises:
a hydrophobic oil core comprising at least one triglyceride, and a hydrophobic drug; and
at least one amphipathic surfactant.

2. The particles of claims 1, wherein the particles have a median diameter of from about 0.5 to about 30 microns, with a standard deviation of the particle diameters of from about 0.1 to about 15 microns.

3. The particles of claim 1, wherein the standard deviation of the particle diameters is from about 0.1 to about 10 microns.

4. The particles of claim 1, wherein the oil core is liquid at ambient temperature.

5. The particles of claim 1, wherein the oil core is solid at ambient temperature.

6. A method of making physiologically active oil-core particles, the method comprising:
a) mixing a hydrophobic solution comprising:
at least one hydrophobic oil material;
a drug, wherein the drug is soluble in the oil material;
at least one amphipathic phospholipid;
a volatile organic solvent; and
optional constituents
with an aqueous solution to form a suspension of physiologically active oil-core particles;
b) removing the volatile organic solvent from the suspension to form a substantially solvent-free suspension of physiologically active oil-core particles.

7. The method of claim 6, wherein the particles have an oil core which is liquid at ambient temperature.

8. The method of claim 6, wherein the particles have an oil core which is solid at ambient temperature.

9. The method of claim 6, wherein the oil material comprises at least one triglyceride having fatty acid chains selected from the group consisting of butyric acid, caproic acid, caprylic acid, capric acid, lauric acid, lauroleic acid, myristic acid, myristoleic acid, pentadecanoic acid, palmitic acid, palmitoleic acid, margaric acid, stearic acid, oleic acid, linoleic acid, linolenic acid, ricinoleic acid, dihydroxystearic acid, licanic acid, eleostearic acid, arachidic acid, eicosenoic acid, eicosapolyenoic acid, behenic acid and erucic acid.

10. The method of claim 9, wherein the oil material comprises at least one triglyceride having fatty acid chains selected from the group consisting of butyric acid, caproic acid, caprylic acid, capric acid, and lauric acid.

11. The method of claim 6, wherein the amphipathic phospholipid is selected from the group consisting of phosphatidic acids, phosphatidylserines, phosphatidylglycerols, phosphatidylinositols, cardiolipins, phosphatidylcholines, phosphatidylethanolamines, and sphingomyelins.

12. The method of claim 6, wherein the optional constituents are selected from the group consisting of diacyl dimethylammonium propanes, acyl trimethylammonium propanes, stearylamine, cholesterol, ergosterol, nanosterol, and esters of those constituents capable of forming esters.

13. The method of claim 6, wherein the aqueous solution comprises water and at least one pharmaceutical excipient.

14. The method of claim 13, wherein the pharmaceutical excipients are selected from the group consisting of amino acids, sorbitol, mannitol and sugars.

15. The method of claim 6, wherein the drug is selected from the group consisting of the oil-phase soluble derivatives of semisynthetic amino glycoside antibiotics, antidiabetics, peptides, antitumor drugs, antineoplastics, alkaloid opiate analgesics, local anesthetics, synthetic anti-inflammatory adrenocortical steroid, antimetabolites, glycopeptide antibiotics, vincaleukoblastines, stathmokinetic oncolytic agents, hormones, cytokines, growth factors.

16. The method of claim 15, wherein the drug is selected from the group consisting of the oil-phase soluble derivatives of paclitaxel, morphine, hydromorphone, bupivacaine, dexamethasone, vincristine and vinblastine.

17. The method of claim 16, wherein the drug is bupivacaine free base, or paclitaxel.

18. The method of claim 6, wherein the particles release drug with a half time of at least 10 hours.

19. The method of claim 18, wherein the particles release drug with a half time of at least 20 hours.

20. The method of claim 18, wherein the particles release drug with a half time of at least 40 hours.

21. The method of claim 6, wherein the particles have a median diameter of from about 0.5 to about 30 microns.

22. The method of claim 6, wherein the particles have a standard deviation of the particle diameter of from about 0.1 to about 15 microns.

23. The method of claim 22, wherein the particles have a standard deviation of the particle diameter of from about 0.1 to about 10 microns.

24. The method of claim 6, wherein the mixing is carried out with a high-speed shear mixer.

25. A method of making physiologically active oil-core particles, the method comprising:
a) mixing a hydrophobic solution comprising:
bupivacaine free base;
tricaprylin;
dioleoylphosphatidylcholine, and dipalmitoylphosphatidylglycerol;
chloroform; and
cholesterol with an aqueous solution comprising 5 mM lysine to form a suspension of physiologically active oil-core particles;
b) removing the chloroform from the suspension to form a substantially chloroform-free suspension of physiologically active oil-core particles.

26. A substantially solvent-free physiologically active suspension made by the method of claim 6.

27. A pharmaceutical composition comprising the substantially solvent-free physiologically active suspension of claim 26.

28. A method of treating, diagnosing, or providing prophylaxis against an undesired condition in an individual, the method comprising administering a pharmaceutical composition according to claim 27.

29. A method of providing anesthesia to an individual in need of anesthesia, by administering a pharmaceutical composition comprising bupivacaine-containing particles made according to the method of claim 6.

30. The method of claim 6, wherein the method is carried out as an aseptic process.

31. A method of making physiologically active oil-core particles, the method comprising:
a) mixing a hydrophobic solution comprising:
at least one hydrophobic oil material;
a drug, wherein the drug is soluble in the oil material;
at least one amphipathic phospholipid; and
optional constituents
with a volatile propellant;
b) allowing volatilization of the propellant to form a substantially solvent-free preparation of physiologically active oil-core particles.

32. The method of claim 31, wherein the volatilization takes place through an orifice of size appropriate to form physiologically active oil-core particles having a median diameter of from about 0.5 to about 30 microns.

33. The method of claim 32, wherein the physiologically active oil-core particles are deposited to contact an oil-immiscible phase.

34. The method of claim 33, wherein the oil-immiscible phase is an aqueous phase.

35. The method of claim 34, wherein the aqueous phase comprises pharmaceutically acceptable adjuvants.

36. The method of claim 31, wherein the propellant is a fluorinated hydrocarbon, or chlorofluorohydrocarbon, and mixtures thereof.

37. The method of claim 31, wherein the volatilization produces an aerosol containing physiologically active oil-core particles in a quantity sufficient to produce physiological effect.

39. The method of claim 38, wherein the drug is paclitaxel.

40. The method of claim 38, wherein the drug is bupivacaine.

41. The physiologically active oil-core particles of claim 1, wherein the hydrophobic drug is paclitaxel, the hydrophobic oil core comprises tributyrin, and the amphipathic surfactants are dipalmitoyl phosphatidylglycerol, dioleoyl phosphatidylcholine and cholesterol.

42. The physiologically active oil-core particles of claim 1, wherein the hydrophobic drug is bupivacaine, the hydrophobic oil core comprises tricaprylin, and the amphipathic surfactants are dipalmitoyl phosphatidylglycerol, dioleoyl phosphatidylcholine and cholesterol.

43. A method of administering physiologically active oil-core particles to a subject, the method comprising: a) formation of an aerosol of the physiologically active oil-core particles of claim 1, b) volatilization of a volatile propellant, and c) allowing contact of the aerosol with the subject.

44. The method of claim 43, wherein the hydrophobic drug is paclitaxel, the hydrophobic oil core comprises tributyrin, and the amphipathic surfactants are dipalmitoyl phosphatidylglycerol, dioleoyl phosphatidylcholine and cholesterol.

45. The method of claim 43, wherein the hydrophobic drug is bupivacaine, the hydrophobic oil core comprises tricaprylin, and the amphipathic surfactants are dipalmitoyl phosphatidylglycerol, dioleoyl phosphatidylcholine and cholesterol.
